(19) United States
(12) Patent Application Publication
(10) Pub. No.: US 2025/0127744 A1
(43) Pub. Date: Apr. 24, 2025

(54) Combination therapy of omega-3, black cumin, manuka honey, Anise, Vitamin-D, Nutmeg in treating COVID-19

(71) Applicants: MOHAMED MEDHAT ABDELWAHAB GAMALELDIN, JEDDAH (SA); SHAIMAA MAHMOUD NASHAT SAYED ABDELHALIM, JEDDAH (SA)

(72) Inventors: MOHAMED MEDHAT ABDELWAHAB GAMALELDIN, JEDDAH (SA); SHAIMAA MAHMOUD NASHAT SAYED ABDELHALIM, JEDDAH (SA)

(73) Assignee: MOHAMED MEDHAT ABDELWAHAB GAMALELDIN, CAIRO (EG)

(21) Appl. No.: 18/395,600

(22) Filed: Dec. 24, 2023

(51) Int. Cl. A61K 31/202; A61K 31/593; A61K 35/644; A61K 36/185; A61K 36/23; A61K 36/71; A61P 31/14; A61P 37/06

(52) U.S. Cl. CPC A61K 31/202; A61K 31/593; A61K 35/644; A61K 36/185; A61K 36/23; A61K 36/71; A61P 31/14; A61P 37/06

(57) ABSTRACT

The present invention relates to treating SARS-CoV-2 infection by concurrent administration of different treatment supplementation through anti-inflammatory, antioxidant, antiviral, and immunoregulation actions. The lower doses of the proposed treatment modalities in the present invention denoted anti-inflammatory, antioxidant, antiviral, and immunoregulation actions. The higher doses of the proposed treatment modalities in the present invention promoted the release the CD4⁺ and CD8⁺ T cell, increasing the phagocytosis activity for viral clearance of SARS-CoV-2 infection. The proposed treatment modalities in the present invention allow the maturation of antibodies against natural SARS-CoV-2 infection.

### CROSS-REFERENCE TO RELATED APPLICATIONS

This application claims the benefits of priority to U.S. Provisional application No. 63/435,263, filed Dec. 25, 2022, the disclosure of which is incorporated herein by reference in its entirety.

### BACKGROUND OF THE INVENTION

COVID-19 infections virus spread worldwide and impact many countries with sever economical sequences. The effective antiviral medication or prophylactic medication for the virus is unavailable until the present date and it takes months or years to discover the effective treatment or test the efficacy of the discovered treatment.

Based on these facts, the human immune system against the virus may have an effective role to regulate the infection and reduce the mortality rate among the infected patients. The present invention aims to explore the available medication/natural supplementation to boost the immunity system of the patients against COVID-19 infections and reduce the mortality rate among infected patients. Methods: a clinical trial (ClinicalTrials.gov ID: NCT04553705) was carried out to investigate the effect of the different treatment modalities on the human immune system and viral replication activity against COVID-19 infection.

Natural supplementations have many reported effects on the human health ranged from immunity boosting to effective antiviral effect. Omeg-3 as an example affect the human health by many mechanisms, e.g., Anti-oxidant, immunity boosting agent. Moreover, Omega-3, Anise, manuka honey exerts an antiviral effect on Flu virus by inhibiting influenza virus replication. Similarly, black cumin exerts an antiviral effect on the replication of old coronavirus and the expression of TRP genes family 3. In addition, Omega-3 regulates the human immunity against bacterial and viral infections. During the past years, many natural sources exert an antimalarial effect with perfect reported results.

Thus, the present disclosure aims to provide promising effective therapeutics with antiviral, antioxidant and immunomodulator effects against COVID-19 infection and exacerbation.

### SUMMARY OF THE INVENTION

The present disclosure fills the gab in treating COVID-19 using effective therapeutic combinations with antioxidant, antiviral, immunomodulator, and anti-inflammatory effects against SARS-CoV-2 infection. The invention's therapeutics demonstrate viral replication inhibitory effect on COVID-19 patients. In addition, the disclosure's therapeutics imply a protective effect on those patients against COVID-19 exacerbation.

The present disclosure claims a combined therapeutic effects through concurrent administration of the disclosure medications. The present disclosure provides an effective treatment method for COVID-19 infection through regulating pro-inflammatory cytokines (prevention of cytokines storm or hypercytokinemia), antioxidant effect to prevent the oxidative stress status and endothelial dysfunction because of the COVID-19 infection, antiviral effect.

Lower doses of the disclosure's medications allow the innate immune response to release the leukocytes and macrophages by regulating the pro-inflammatory cytokines through the anti-inflammatory cytokines.

Higher doses the disclosure's medications allow the adaptive immune response to release CD4+ and CD8+ T cells to clear the viral infection by increasing the phagocytosis activity.

The disclosure's medications prevent the complication of mild COVID into severe COVID by preventing cytokines storm.

The Omega-3, black seed, and nutmeg supplementations showed antiviral effect against COVID19 infection at lower doses.

The disclosure's medications allow the natural infection to dominate the early stage of the infection by regulating the pro-inflammatory cytokines and prevent cytokines storm at the lower doses, allowing the maturation of antibodies against COVID19 infection through the delayed adaptive immune response.

The Concurrent administration of the medications showed combined therapeutic effect through regulating the pro-inflammatory cytokines at the lower doses and increase the phagocytosis and viral clearance at the higher doses.

The Concurrent administration of the medication showed combined antioxidant/anti-hemolysis effect against the oxidative stress and endothelial dysfunction secondary to COVID19 infection.

The Concurrent administration of omega-3, black seed, and nutmeg showed combined antiviral effect against COVID19 infection.

The concurrent administration of the disclosure's medications promotes the release of CD4+T and CD8+ T cells for viral clearance and clinical improvements.

### DETAILED DESCRIPTION OF THE INVENTION

The present disclosure provides an effective treatment modality for the human immunity system. A proposed randomized clinical trial investigated the efficacy of different treatment modalities on the human immunity system against COVID-19 infection. The associated clinical trial with the present invention was NCT04553705 at ClinicalTrials.gov. The following are the proposed mechanism of actions of the proposed interventions against COVID-19 infection.

A randomized-clinical trial was performed on 25 SARS-CoV-2 infected patients receiving the proposed treatment modalities in the present invention with 25 control patients receiving the conventional treatment protocol of SARS-CoV-2 infection.

The proposed treatment modalities in the present invention were as follows: omega-3 supplementation 350 mg (12 mg/kg/day) Eicosapentaenoic acid (EPA) and 250 mg (6 mg/kg/day) docosahexaenoic acid (DHA), 500 mg of 3% Thymoquinone-black cumin, 450 mg Anethol capsules, Vitamin-D tablet 10 μg/day, Manuka honey tablet MGO-400 (Methylglyoxal-400)=344 mg (12 mg/kg/day), 600 mg/day myristicin capsules.

The concurrent administration of the proposed treatment modalities in the present invention referred to initially administrating the omega-3, black cumin, and myristicin capsules, followed by Vitamin-D, Anethol, Manuka honey supplementation after one hour from the initial administration.

The lower doses of proposed treatment modalities in the present invention were one tablet/capsule of each supplement per day for the first 10 days post- SARS-CoV-2 natural infection.

The higher dose of the proposed treatment modalities in the present invention were two tablet/capsule of each supplement per day for the subsequent 20 days post- SARS-CoV-2 natural infection.

The lower doses of the proposed treatment modalities in the present invention denoted anti-inflammatory, antioxidant, and immunoregulation effects and regulate the pro-inflammatory cytokines, preventing the cytokines storm against the early stage of SARS-CoV-2 natural infection.

The higher doses of the proposed treatment modalities in the present invention denoted antiviral and adaptive immune response stimulator against the late stage of SARS-CoV-2 natural infection.

The concurrent administration of the proposed treatment modalities in the present invention denoted a combined therapeutic effect against SARS-CoV-2 natural infection.

The proposed mechanism of action of omega-3 supplementation implied on the oxidative stress status which is the main source of ROC that weak the immunity system. PUFAs in omega-3 supplements limits the excessive inflammation and improve the immune response by migration and activation of phagocytic cells and T-cells into infected cells, Then initiation of adaptive immune response by recognition of antigen by B-lymphocyte. Omega-3 exerts antioxidant effect on oxidative stress status that promote immunity. The antiviral effect of omega-3 supplementation mediated through lipid mediators Protectin D1 that inhibits virus replication.

The proposed mechanism of action of Thymoquinone in the black cumin capsules implied through antihypertensive action by reducing the Angiotensin converting enzyme activity in COVID-19 patients. Moreover, TQ denotes immunomodulator and antiviral effect against SARS CoV-2 replication activity.

The proposed mechanism of action of Manuka honey supplementation is based on the main active ingredient, methylglyoxal, through anti-inflammatory, antioxidant and immunoregulator effects.

The proposed mechanism of action of Vitamin-D tablet was the antihemolytic and anti-inflammatory effects.

The proposed mechanism of action of Anethol capsules was the antioxidant and anti-inflammatory effects.

The proposed mechanism of action of myristicin capsules was the antiviral effect.

The clinical/laboratory parameters were investigated in the present invention were clinical and symptomatic improvement after 30 days post SARS-CoV-2 natural infection, body temperature after 15 days, CT scan for lung inflammation after 30 days, PCR level every 15 days for 2 months, oxygen saturation every 5 days for one month, LDH serum level every 5 days for one month, C-reactive protein every 5 days for one month, Leukocytes count every 5 days for one month, and proinflammatory cytokines (IL-1β, IL6, IL-8, IFN-γ, TNF-α).

The biochemical parameter (LDH, C-reactive protein, leukocytes) were measured using hematology analyzer (LH-series and AU-series Beckman coulter commercial kits, Beckman Coulter Inc., Fullerton, CA, USA.

The proinflammatory cytokines (IL-1β, IL6, IL-8, IFN-γ, TNF-α) were measured using Cytometric Bead Array Kits (BD Biosciences Pharmingen, San Diego, CA) using flow cytometry.

The CD4+ and CD8+ T cell were measured using four-color flow cytometry and the commercially available Multitest IMK® kit (Becton Dickinson, USA).

### Results

The proinflammatory cytokines (IL-1β, IL6, IL-8, IFN-γ, TNF-α) were significantly reduced by 25% from baseline levels at admission after 5 days of treatment with the proposed combined treatment in the present invention.

The CD4+ and CD8+ T cell were significantly increased after 15 days with treatment.

PCR level was significantly reduced by 30% from the baseline level at patient admission.

CT-scan of the left and right lung showed reduced inflammation by 20% from the baseline findings at patient admission.

Oximetry testing showed 92% oxygen saturation after 12 days with the proposed combined treatment.

The present disclosure claimed the following:

1. A combined treatment modalities is proposed for treating severe acute respiratory syndrome coronavirus 2 (SARS-CoV-2) by concurrent administration of omega-3, Black cumin, Anise, Manuka honey, Vitamin-D, and nutmeg supplementations at different therapeutic doses.

2. The proposed treatment modalities in the present disclosure prevent complication of mild COVID into severe COVID by preventing cytokines storm (hypercytokinemia).

3. The proposed treatment modalities in the present disclosure denoted anti-inflammatory, antioxidant, and immunoregulator effect against the SARS-CoV-2 natural infection.

4. The proposed treatment modalities in the present disclosure promote the release of CD4+T and CD8+ T cells, allowing for viral clearance and clinical improvements in SARS-CoV-2 infected patients.

5. The lower doses of the proposed treatment modalities in the present disclosure allow the innate immune response to release the leukocytes and macrophages by regulating the pro-inflammatory cytokines through the anti-inflammatory cytokines.

6. The higher doses of the proposed treatment modalities in the present disclosure allow the adaptive immune response to release CD4+ and CD8+ T cells to clear the viral infection by increasing the phagocytosis process.

7. The concurrent administration of Omega-3, black seed, and nutmeg supplementation showed antiviral effect against COVID19 infection at lower doses.

8. The proposed treatment modalities in the present disclosure allow the natural infection to dominate the early stage of the infection by regulating the pro-inflammatory cytokines and prevent cytokines storm at the lower doses, allowing the maturation of antibodies against COVID19 infection through the delayed adaptive immune response.

9. The Concurrent administration of the proposed treatment modalities in the present disclosure showed combined therapeutic effect through regulating the pro-inflammatory cytokines at the lower doses and increase the phagocytosis and viral clearance at the higher doses.

10. The Concurrent administration of the proposed treatment modalities in the present disclosure showed combined antioxidant/anti-hemolysis effect against the oxidative stress and endothelial dysfunction secondary to SARS-CoV-2 infection.
